(19) United States
(12) Patent Application Publication
(10) Pub. No.: US 2013/0149383 A1
(43) Pub. Date: Jun. 13, 2013

(54) SUSTAINED RELEASE PARTICLE FORMULATIONS OF GUAIFENESIN

(71) Applicants: Cory Berkland, Lawrence, KS (US); Milind Singh, Mission, KS (US)

(72) Inventors: Cory Berkland, Lawrence, KS (US); Milind Singh, Mission, KS (US)

(21) Appl. No.: 13/712,621

(22) Filed: Dec. 12, 2012

Related U.S. Application Data
(60) Provisional application No. 61/569,664, filed on Dec. 12, 2011.

(51) Int. Cl. A61K 31/09

(52) U.S. Cl. CPC A61K 31/09
USPC 424/490; 424/400; 514/718

(57) ABSTRACT

Sustained release particle formulations formed from a hydrophobic wax matrix and guaifenesin.

### CROSS REFERENCE TO RELATED APPLICATIONS

This application claims priority to U.S. Provisional Application No. 61/569,664, filed Dec. 12, 2011, which is incorporated herein by reference.

### BACKGROUND

Modified or sustained release pharmaceutical dosage forms have long been used to optimize drug delivery and enhance patient compliance, especially by reducing the number of doses of medicine the patient must take in a day. The use of sustained release dosage forms has increased due to dosing convenience and potentially reduced adverse effects. Multiple-unit sustained release dosage forms have been used for the delivery of therapeutic agents due to their inherent clinical advantages over single-unit dosage forms. These dosage forms spread out uniformly in the gastrointestinal tract and potentially reduce the risk of local irritation and dose dumping, which are often seen with single-unit dosage forms.

Well known mechanisms by which a dosage form (or drug delivery system) can deliver drug at a modified rate (e.g. sustained or delayed release) include diffusion, erosion, and osmosis. An important objective of modified release dosage forms is to provide a desired blood concentration versus time profile for the drug. Fundamentally, the pharmacokinetic profile for a drug is governed by the rate of absorption of the drug into the blood, and the rate of elimination of the drug from the blood. To be absorbed into the blood (circulatory system), the drug must first be dissolved in the gastrointestinal fluids. For those relatively rapidly absorbed drugs whose dissolution in gastrointestinal fluids is the rate limiting step in drug absorption, controlling the rate of dissolution (i.e. drug release from the dosage form) allows the formulator to control the rate of drug absorption into the circulatory system of a patient.

### SUMMARY

The present disclosure generally relates to particles for sustained delivery of guaifenesin. More particularly, the present disclosure provides, according to certain embodiments, compositions comprising particles, the particles comprising guaifenesin, a hydrophobic wax matrix, a stabilizer, and a release modifier; wherein the particles are substantially free of water; and wherein the particles have a diameter of from about 20 μm to about 500 μm

The features and advantages of the present invention will be readily apparent to those skilled in the art. While numerous changes may be made by those skilled in the art, such changes are within the spirit of the invention.

### DRAWINGS

FIG. 1 is a table and graph depicting the relationship between active ingredient concentration and HPLC area.

FIG. 2 is a graph depicting the relationship between time and the release of guaifenesin.

FIG. 3 is a diagram showing a procedure used to form particles of the present disclosure, according to one embodiment.

FIG. 4 is a graph showing particle size distribution results.

FIG. 5 is a graph showing particle size distribution results.

FIG. 6 is a graph depicting the relationship between time and the release of guaifenesin.

FIG. 7 is a graph depicting the relationship between time and the release of guaifenesin.

FIG. 8 is a graph depicting the relationship between time and the release of guaifenesin.

While the present disclosure is susceptible to various modifications and alternative forms, specific example embodiments have been shown in the figures and are described in more detail below. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present invention relates to particles for sustained delivery of guaifenesin. Guaifenesin (3-(2-methoxyphenoxy)-1,2-propanediol) is a highly water soluble drug and is used as an expectorant in the symptomatic treatment of coughs associated with common cold and other respiratory symptoms. Guaifenesin has a typical plasma half-life of approximately one hour. Guaifenesin is available in two general formulations, immediate release and sustained release. With an immediate release formulation, patients take guaifenesin once every four hours to maintain adequate bioavailability. This results in a rapid increase and a rapid decrease of in the blood concentrations of guaifenesin, meaning that the patient is provided with a short duration within the therapeutic window of the drug for optimum therapy. Sustained release formulations of guaifenesin, on the other hand, may provide a longer duration within the therapeutic window, but also may suffer from irregular dissolution and/or dose profiles. In certain embodiments, the present disclosure provides particles that provide dissolution and/or dose profiles suitable for sustained delivery of guaifenesin, as well as formulations comprising such particles.

The present disclosure provides, according to certain embodiments, compositions comprising particles, the particles comprising guaifenesin, a hydrophobic wax matrix, a stabilizer, and a release modifier; wherein the particles are substantially free of water or other aqueous solvent; and wherein the particles have a diameter of from about 20 μm to about 500 μm. In certain embodiments, the particles may be configured to have sustained release of the guaifenesin over a period of 8 hours or more.

The guaifenesin active ingredient is disposed within the hydrophobic wax matrix. The guaifenesin may be homogenously dispersed within hydrophobic wax matrix via a molten or solubilized form. The guaifenesin also may be dispersed within hydrophobic wax matrix as small particulates. Alternatively, the guaifenesin may be disposed substantially within the hydrophobic wax matrix in a core-shell configuration in which the hydrophobic wax matrix is the shell. As opposed to prior sustained release formulations of guaifenesin, the particles of the present disclosure are substantially free of water or other aqueous solvent.

The guaifenesin may be present in the particles in an amount in the range of from about 20% to about 60%, 25% to about 50%, or 30% to about 40% by weight of the particles. In some specific embodiments, the amount of guaifenesin may be 32% of the weight of the particles. The guaifenesin may be present in the particles in an amount sufficient to provide any suitable dosage. In some embodiments, the guaifenesin may be present in the particles in an amount sufficient to provide a daily dose of between 600 mg and 1200 mg. In one embodiment, guaifenesin may be present in the particles in an amount sufficient to provide a daily dose of 950 mg.

As noted above, the particles of the present disclosure are formed from a hydrophobic wax matrix. The hydrophobic wax matrix may be any wax-like material suitable for use with guaifenesin and suitable for administration to a patient. Examples of suitable hydrophobic waxes include, but are not limited to, ceresine wax, beeswax, ozokerite, microcrystalline wax, candelilla wax, montan wax, carnauba wax, paraffin wax, cauassu wax, Japan wax, and Shellac wax.

The hydrophobic wax matrix may be present in the particles in an amount in the range of from about 30% to about 80%, about 30% to about 60%, about 35% to about 70%, or about 40% to about 50% by weight of the particle. In other embodiments, the hydrophobic wax matrix may be present in the particles in an amount of about 55%, 57%, or 65% by weight of the particle. In other embodiments, the wax may be present in the particles in an amount sufficient to provide sustained release of the guaifenesin over a period ranging between about 1 hour to about 12 hours. For example, the hydrophobic wax matrix may be present in the particles in an amount sufficient to provide sustained release of the hydrophilic active ingredient over a period of about 6 hours, 8 hours, 10 hours, 12 hours, or more than 12 hours.

In general, the particles of the present disclosure have a mean particle size diameter of from about 20 μm to about 500 μm. In certain embodiments, the particles have a mean particle size diameter of from about 50 μm to about 300 μm. In other embodiments, the particles may be substantially monodisperse with a relatively narrow particle size distribution with a 25% or less standard deviation from the mean particle size. In other embodiments, the particles may be substantially monodisperse with a relatively tight particle size distribution with 10-15% standard deviation from the mean particle size. In a specific embodiment, the mean particle diameter may range from 150 μm to 250 μm. In some embodiments, two or more populations of substantially monodisperse particle sizes may be used. The particular particle size, or mixture of particle sizes, will depend on the desired release profile.

In some embodiments, relatively tight particle size distributions may be preferred. Such particle size distributions benefit from the lack of “fines.” Particle fines are small particles left over from a manufacturing process. Their small effective surface area results in faster dissolution rates. As used herein, the term “fines” refers to particulates having a particle size at or below 10% of the mean particle size diameter. Accordingly, formulations having particle fines are not substantially monodisperse and may not provide the desired dissolution properties and/or bioavailability.

As noted above, the particles of the present disclosure comprise a stabilizer. The stabilizer may improve the properties of the hydrophobic wax matrix and provide improved stability of the particles over time, as well as improved dissolution profiles. Changes in particles can occur over time that affect the particle's performance. Such changes include physical, chemical, or dissolution instability. These changes are undesirable as they can affect a formulation's shelf stability, dissolution profile, and bioavailability of the active ingredient. For example the hydrophobic wax matrix or active ingredient may relax into a lower energy state, the particle may become more porous, and the size and interconnectivity of pores may change. Changes in either the active ingredient or hydrophobic wax matrix may affect the performance of the particle. The present disclosure is based, at least in part, on the observation that a stabilizer added to the hydrophobic wax matrix improves the stability and performance of the particles of the present disclosure. By way of explanation, and not of limitation, it is believed that the stabilizer interacts with the hydrophobic wax material making it resistant to physical changes. Accordingly, the particles of the present disclosure comprise a stabilizer. Examples of suitable stabilizers include but are not limited to, cellulose, ethyl cellulose, hydroxyproylmethyl cellulose, microcrystalline cellulose, cellulose acetate, cellulose phthalate, and methyl cellulose and mixtures thereof. Stabilizers may be used alone or in combination. The stabilizer may be present in the particles in an amount from about 0.1% to about 5%, about 0.5% to about 2.5%, and about 1% by weight of the particle.

The particles of the present disclosure also comprise a release modifier. The present disclosure is also based on the observation that a release modifier improves the performance of hydrophobic wax matrix particles particularly during the later stages of the active ingredient's release. The release modifier is believed also to interact with the stabilizer (e.g., improve the stabilizer's solubility) to facilitate preparation of the particles. It is also believed that the release modifier may adjust the relative hydrophobicity of the hydrophobic wax material. Examples of suitable release modifiers include but are not limited to, stearic acid, sodium stearate, magnesium stearate, glyceryl monostearate, cremophor (castor oil), oleic acid, sodium oleate, lauric acid, sodium laurate, myristic acid, sodium myristate, vegetable oils, coconut oil, mono-, di-, tri-glycerides, stearyl alcohol, span 20, and span 80. Release modifiers may be used alone or in combination. For example, in certain embodiments, the release modifier may be a combination of stearic acid and glyceryl mono stearate. The release modifier may be present in the particles in an amount from about 0.5% to about 10%, about 1% to about 5%, about 2.5% to about 5%, and about 2% by weight of the particle.

In some embodiments, the particles of the present disclosure may further comprise pharmaceutically acceptable inactive ingredients. The term “pharmaceutically acceptable,” when used in connection with the pharmaceutical compositions of the invention, refers to molecular entities and compositions that are physiologically tolerable and do not typically produce untoward reactions when administered to a human. For example, “pharmaceutically acceptable” may refer to inactive ingredients approved by a regulatory agency of the Federal or a state government or listed in the U.S. Pharmacopeia or other generally recognized pharmacopeia for use in animals, and more particularly in humans. Examples of inactive ingredients that may be included in particles or formulations of the present disclosure include but are not limited to, buffers, preservative, suspending agents, dyes, antioxidants, surfactants, and the like.

In some embodiments, the particles of the present disclosure may comprise an additional layer disposed on the surface of the particle. Such layers may be used to reduce or delay the release of active ingredient from the particles or to mask the taste of the active ingredient. The additional layer may be a coating applied to the surface of the particle. Such coating may be formed from any material capable of being applied to a pharmaceutical composition. Coatings may be applied to the particles using techniques known in the art such as, for example, Wurster coating and techniques described in U.S. Pat. Nos. 6,669,961, 7,309,500, and 7,368,130, all of which are incorporated by reference.

Examples of suitable materials that may be applied to the surface of the particle to, among other things, reduce or delay the release of active ingredient from the particles include, but are not limited to, polymethacrylates, materials from Eudragit®, Surelease® or Kollicoat® series, and cellulose materials (e.g., ethyl cellulose, hydroxypropylmethyl cellulose).

Examples of suitable materials that may be applied to the surface of the particle to, among other things, mask the taste of the active ingredient include, but are not limited to, mono-, di-, or polysaccharides, sugar alcohols, or other polyols such as lactose, glucose, raffinose, melezitose, lactitol, mannitol, maltitol, trehalose, sucrose, and starch; ethyl cellulose, methyl cellulose, hydroxypropyl cellulose, hydroxypropyl methylcellulose, hydroxybutyl methylcellulose, cellulose propionate, cellulose acetate propionate, cellulose acetate butyrate, cellulose acetate phthalate, carboxymethyl cellulose, cellulose triacetate, polymethyl methacrylate, polyethyl methacrylate, polyphenyl methacrylate, polymethyl acrylate, polyisopropyl acrylate, polyisobutyl acrylate, polyisobutyl methacrylate, polyhexyl methacrylate, polyphenyl methacrylate, polyvinyl acetate, polyvinyl isobutyl ether, polyvinyl alcohol, polyethylene terephthalate, polyethylene oxide, polyethylene glycol, polyethylene, polypropylene, polyoctadecyl acrylate, polyvinyl chloride, and polyvinyl pyrrolidone.

In one embodiment, the additional layer may comprise the hydrophobic wax matrix, stabilizer, release modifier, and optionally an active ingredient (e.g., guaifenesin). When included, guaifenesin may be present the same or different amounts than is present in the remainder of the particle. Such additional layer may further include a coating as described above.

In certain embodiments, the entire dose of the guaifenesin may be provided by guaifenesin in the particle. In other embodiments, the particle provides a partial dose of the guaifenesin. In such embodiments, the remainder of the dose may be included in a composition apart from the particles. For example, guaifenesin may be included in a liquid vehicle in which the particles are suspended.

As mentioned above, in certain embodiments the particles may be configured to have sustained release of the guaifenesin over a period anywhere between about 1 hour to about 12 hours, or more. The sustained release results from, at least in part, disposing the guaifenesin in the hydrophobic wax matrix. Accordingly, in certain embodiments, the hydrophobic wax matrix layer may be increased or decreased depending on the particular release characteristics desired. In addition, more than one hydrophobic wax matrix layer may be used to achieve the particular sustained release desired. In other embodiments, the size of the particles, or a mixture of differently sized particles, may be chosen depending on the particular release characteristics desired.

In certain embodiments, the particles may further comprise a densifier. A densifier may used to increase the density of a particle. For example, a densifier may be used to make a particle heavier so that it will approach or be closer to the density of a liquid vehicle in which the particles may be suspended. Examples of suitable densifiers include, but are not limited to, titanium dioxide, calcium phosphate, and calcium carbonate. In one embodiment, the one or more densifiers may be present in the particles in an amount in the range of from about 0% to about 40%, 5% to about 30%, 10% to about 25%, and 15% to about 20% by weight of the particles.

In certain embodiments, the particles of the present disclosure are stable. Stability is an important consideration for pharmaceutical formulations. For solid dosage forms, like the particles of the present disclosure, stability may be measured with reference to dissolution. Dissolution testing is an in vitro method that characterizes how an API is extracted out of a solid dosage form. It can indicate the efficiency of in vivo dissolution. Dissolution can be measured using standard protocols. As used herein, the term stable or stability refers to particles of the present disclosure that show a standard deviation of 10% or less in the release profile at any given time point during the course of dissolution when placed at 40° C. for up to at least 4 weeks as measured by United States Pharmacopeia (USP) II dissolution.

The present disclosure also provides formulations comprising particles of the present disclosure. Such formulations may be in the form of a suspension of particles, tablets, capsules, or any other suitable means of formulating particulates into dosage forms suitable for administration to a patient. In certain embodiments, formulations of the present disclosure may further comprise a liquid vehicle. As mentioned above, the liquid vehicle may comprise guaifenesin, which may be in dissolved or suspended form. The liquid vehicle may be aqueous based and may include any component suitable for use in a liquid vehicle as is well known in the art. For example, the liquid vehicle may include one or more of a filler, a sugar, a salt, a viscosity modifier, colorants, preservatives, and the like.

In general, the particles of the present disclosure may be made using methods comprising melting the particle components together followed by particle fabrication. Such procedures may be performed in essentially a single step and without the use of water or other aqueous solvent. This has several advantages. For example, the resulting particles are dry and ready for further processing or formulation and the resulting particles. Similarly, the resulting particles are substantially free of water, which may improve the stability of the active ingredient. The lack of water in the particles means that pores or voids in the particle do not form from evaporation of water droplets. Because the particles can be made without water or an emulsion step, the particles can be formed more efficiently and with fewer manufacturing artifacts. These procedures also allow higher concentrations of active ingredient to be loaded in the hydrophobic wax matrix. Similar, the procedures of the present disclosure offer encapsulation efficiencies for the active reaching greater than 90%. Additionally, the procedure provides particles substantially free of fines, the presence of which can adversely affect the active's release profile.

In certain embodiments, the particles of the present disclosure may be made by melting the components together followed by particle fabrication. For example, particles of the present disclosure may be made by adding to a preheated vessel the following components: a hydrophobic wax, a releasing agent, and the active ingredient (e.g., guaifenesin). The components are then melted and allowed to equilibrate at a temperature of about 120° C. The stabilizer may then be added and allowed to dissolve into the mixture. The temperature of the resulting mixture is then allowed cool to between about 85° C. to about 95° C. for particle fabrication. The particle fabrication may use the techniques disclosed in techniques described in U.S. Pat. Nos. 6,669,961; 7,309,500; and 7,368,130, all of which are incorporated by reference. Particle fabrication also may use other techniques known in the art such as, for example, a spinning disk atomizer, centrifugal coextrusion, prilling, spray congealing, spray cooling, melt atomization, and melt congealing.

In another embodiment, the particles of the present disclosure may be using a similar melting procedure in which the releasing agent and stabilizer are introduced into a preheated vessel and allowed to solubilize at a temperature of about 120° C. (e.g., for about 5-20 minutes). In operation, the releasing agent in its molten form may be used to substantially solubilize the stabilizer. This mixture's temperature is then reduced to between about 100° C. to about 110° C. and the hydrophobic wax and active ingredient (e.g., guaifenesin) are then added. The resulting combination is mixed well (e.g., 1 hour) while the temperature is maintained between about 100° C. to about 110° C. After mixing, the temperature of the mixture is allowed cool to between about 85° C. to about 95° C. before starting the particle fabrication using techniques described above.

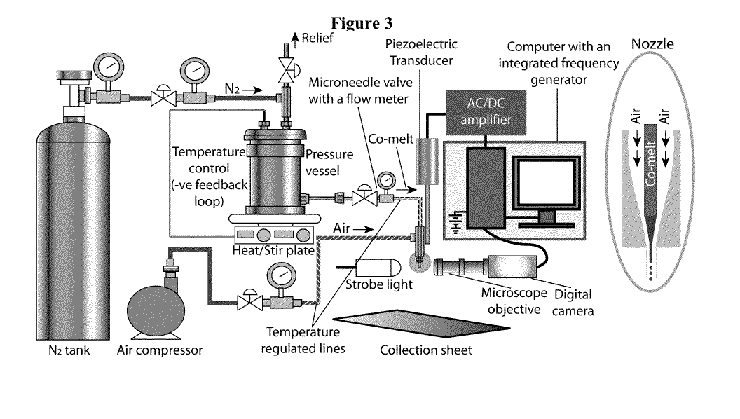

A schematic showing one example of a procedure for making particles of the present disclosure is shown in FIG. 3.

In certain embodiments, after particle fabrication the particles may be treated to reduce the occurrence of pores on the surface of the particle. In this approach, the particles are allowed to cool to room temperature (e.g., over about 6 to 24 hours) then exposed to a brief heat treatment at, for example, 65° C. or other temperature slightly lower than the melting temperature of the formulation ingredient with minimum melting temperature. Such heat treatment may reduce the occurrence of a burst of active ingredient in the release profile of the particle.

To further illustrate various illustrative embodiments of the present disclosure, the following examples are provided.

### EXAMPLES

The examples herein are illustrations of various embodiments of this invention and are not intended to limit it in any way.

### Example 1

### Particles Containing Guaifenesin

An exemplary formulation was developed to match the Mucinex™ Max 1200 mg dose. The particles for this formulation were formed with 45.5% (by weight) candelilla, 32% guaifenesin, 2.5% filler, 10% TiO₂ densifier, 10% CaCO₃ densifier, which corresponds to an amount per dose/day (based on Mucinex™ Max dose) of 1351 mg, 1200 mg (950 mg in the particles and 250 mg in the vehicle), 74 mg, 297 mg, 297 mg, respectively. The vehicle included 90 g/100 mL high fructose corn syrup, 36 g/100 mL Neosorb 70/02 (Neosorb 70% sorbitol solution), 10 g/100 mL glycerin, 5% wt/wt SCD (sodium citrate dehydrate), 3% wt/wt NaCl, 2.5% wt/wt MMSP (monobasic monohydrate sodium phosphate) 1% wt/wt sodium acetate.

Equilibrium Solubility Determination Protocol.

The objective of this example was to determine the drug loading in the particles using “HPLC protocol”. About 500 mg of guaifenesin was added into 20 ml of a liquid vehicle. The suspend solution were then shaken and then placed at 40° for two days. The supernatant was then filtered off using a syringe with a 0.45 μm filter, and diluted so that the absorbencies fell within the UV (25-fold dilution). The diluted clear solutions were equilibrium solubility samples, termed as “samples” in the “HPLC protocol’. The “HPLC protocol” was then followed (See below).

Loading Determination Protocol.

The objective of this example was to determine the drug loading in the particles using “HPLC protocol.” About 40 mg of particles (assuming about a 32% theoretic drug loading) were added to 20 mL of DI water in a scintillation vial. The vial was heated to around 90-110° C. using a heat/stir plate. One the wax melted, the vials were cooled down and the liquid was filtered using a syringe with a 0.45 μm filter. The collected clear solutions were loading samples, termed as “samples” in the “HPLC protocol.” The “HPLC protocol” was then followed (See below).

USP II Dissolution Protocol.

The objective of this example was to determine the dissolution profile of particles over a period of 12 h, and compare the dissolution profile to Mucinex™ Max.

A liquid vehicle (20 mL) was transferred to glass scintillation vials and 250 mg of pure guaifenesin was added to each vial. This drug suspension was vortexed for 2-3 min at 500 rpm and was then left in an environmental chamber at 40° C. for 48 h to saturate the liquid with the immediate release (IR) guaifenesin.

The dissolution study was performed using a Vanderkamp 600 six-spindle dissolution tester with Hanson 900 mL dissolution jars. The temperature of the medium was maintained at (37±1)° C. The distance between the impeller and dissolution jar bottom was fixed at 2.5 cm, and the impeller rotation speed was fixed at 75 rpm. Mucinex™ Max was used as a positive reference control group. Drug loading in Orbis microspheres was determined (see Example 2), which was found to be 32%.

The amount of particles used for each group was selected to keep the drug load constant, and was matched to the drug load of the control group (i.e., 1200 mg). Since 250 mg guaifenesin is present in the liquid vehicle in the IR form, the sustained release (SR) contribution from the particles was fixed at 950 mg. This equates to 2.97 g of particles per vessel (with 32% drug loading in the particles). Immediately before the dissolution testing, 2.9 g of particles were mixed with the liquid IR formulation (which contained 250 mg of guaifenesin in the IR form) in the same scintillation vials. The particle-liquid formulation was transferred to the dissolution vessel. 880 ml of 0.1 N HCl with 0.05% (v/v) of Tween 80 was added to each vessel (Note: the dissolution solution was pre-equilibrated at 37° C. Also, 50 mL of 880 mL dissolution solution was used to wash each scintillation vial to ensure complete recovery of the particles from the scintillation vial). The temperature of the medium was maintained at 37±1° C. For each sampling, 1.0 ml of dissolution media was sampled at 1, 2, 6, and 12 h, which were then analyzed using HPLC.

HPLC Protocol.

The objective of this example was to analyze the samples using HPLC and determine the drug loading using a standard curve for the drug.

A 20 mL of stock solution of the drug was prepared in DI water at a concentration of 1 mg/mL. The solution was left at room temperature for 5 min to get the drug dissolved. The stock solution was appropriately diluted to get several concentrations ranging from 0.1 mg/mL to 1 mg/mL (See FIG. 1). Samples were prepared by appropriately diluting the samples collected using “USP dissolution protocol” to ensure that the drug concentration level falls within the range of the standard curve (e.g., 2× dilution).

HPLC was prepared by first washing the column with the wash buffer (acetonitrile:water 50:50 (v/v)) for 10 min. The HPLC was then primed with the mobile phase based on the following conditions: injection volume=25 μL, flow rate=1.0 ml/minutes, detector UV at 254 nm, mobile phase (620:390) 0.023 M sodium dodecyl sulfate and 0.02 M ammonium nitrate:acetonitrile, and retention time=2.2 minutes. The standards/samples were then run. After the run was over, the column was washed with the wash buffer. The mobile phase was stored in refrigerated condition until used. During the HPLC area determination analysis, to ensure that the baseline was correctly placed, the “Baseline now” was set to 2 min, which ensured a correct baseline for the retention period of 2.2-2.3 min.

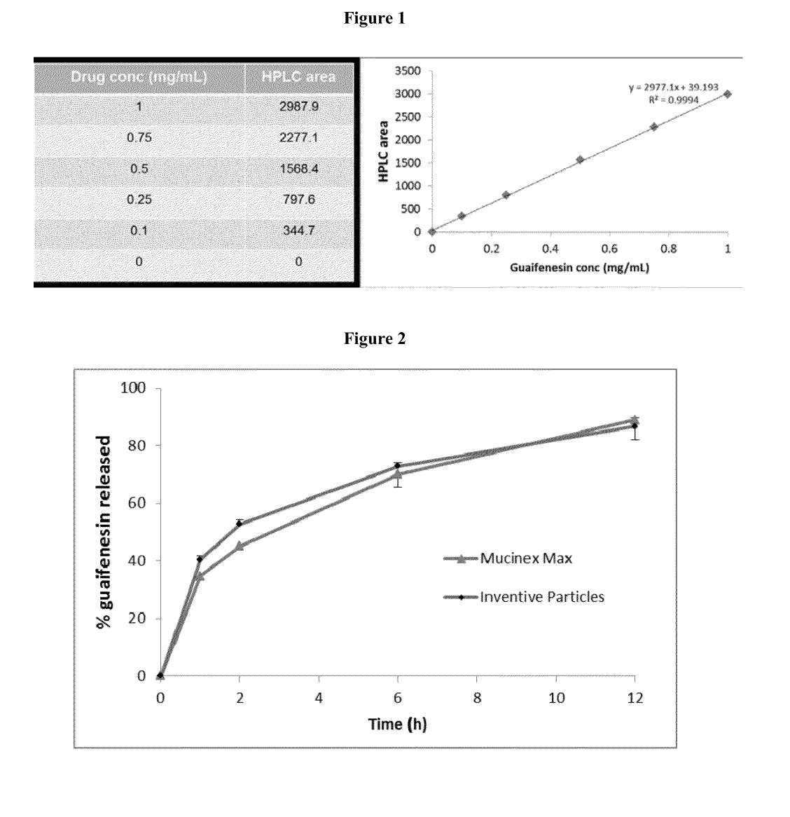

The results of this example showed an equilibrium solubility of the liquid vehicle 9.59±0.34 mg/mL (n=3). The drug loading in the particles was found to be about 32%. The USP II dissolution test results (See FIG. 2) were as follows:

| | | | Inventive |
|---|---|---|---|
| Time (h) | Target % release | Mucinex ™ Max* | formulation** |
| 1 | <45% | 34.8 ± 1.1% | 40.4 ± 1.3% |
| 2 | 40%-55% | 45.1 ± 1.0% | 52.7 ± 1.5% |
| 6 | 62%-80% | 70.0 ± 4.3% | 72.9 ± 1.3% |
| 12 | >85% | 88.9 ± 6.8% | 86.6 ± 3.0% |

*Mean ± standard deviation (n = 6)
**Mean ± standard deviation (n = 3)

### Example 2

### Particles Containing Guaifenesin

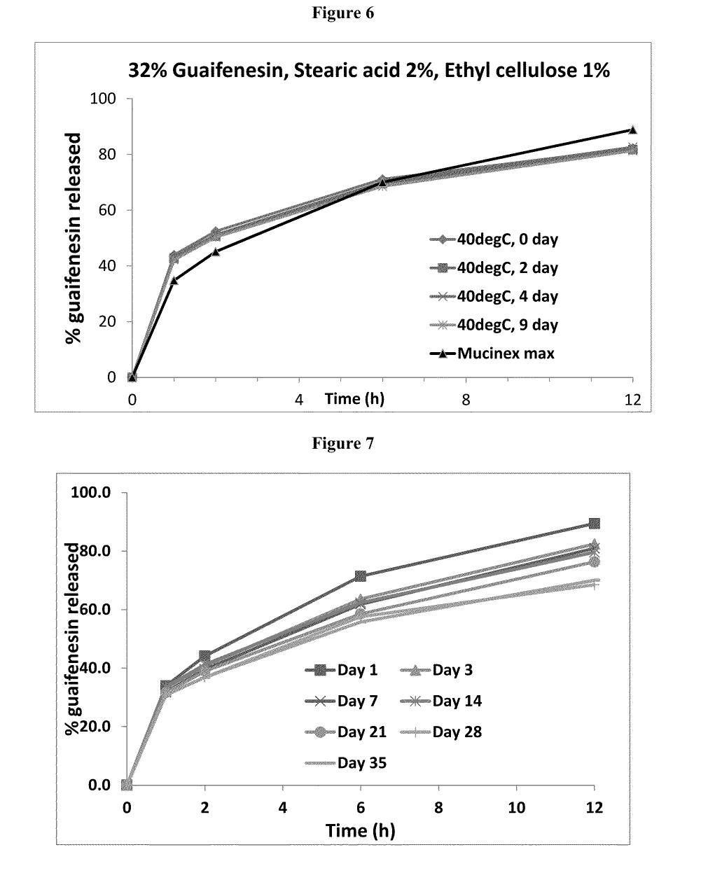

An exemplary guaifenesin particle was formed with 65% carnauba wax, 2% stearic acid, 32% guaifenesin, and 1% ethyl cellulose. The release of guaifenesin from these particles was measured at 40° C. over 21 days as follows. Samples were kept at 40° C. in an environmental chamber in closed glass vials for the duration of the study. At each time point, samples were taken out from the incubator, allowed to cool down to room temperature followed by USP II dissolution study. The results are shown in FIG. 6 and Table 2.

TABLE 2

| | Percent Release | | | |
|---|---|---|---|---|
| Time (h) | Day 0 | Day 7 | Day 14 | Day 21 |
| 0 | 0.0 | 0.0 | 0.0 | 0.0 |
| 1 | 44.0 | 42.7 | 43.0 | 42.1 |
| 2 | 52.5 | 50.7 | 51.2 | 50.3 |
| 6 | 71.2 | 69.6 | 70.0 | 68.5 |
| 12 | 82.2 | 81.7 | 82.7 | 81.3 |

### Example 3

### Particles Containing Guaifenesin

An exemplary guaifenesin particle was formed with 57% carnauba wax, 10% stearic acid, 32% guaifenesin, and 1% ethyl cellulose. The release of guaifenesin from these particles was measured at 40° C. over 35 days, as described above. The results are shown in FIG. 7 and Table 3.

TABLE 3

| | Percent Release | | | | | |
|---|---|---|---|---|---|---|
| Time (h) | Day 0 | Day 7 | Day 14 | Day 21 | Day 28 | Day 35 |
| 0 | 0.0 | 0.0 | 0.0 | 0.0 | 0.0 | 0.0 |
| 1 | 33.9 | 32.3 | 31.7 | 33.6 | 31.6 | 31.0 |
| 2 | 44.1 | 40.7 | 39.6 | 41.3 | 38.9 | 36.8 |
| 6 | 71.4 | 63.6 | 61.8 | 62.3 | 58.5 | 57.6 |
| 12 | 89.4 | 82.4 | 80.9 | 79.6 | 76.3 | 68.5 |

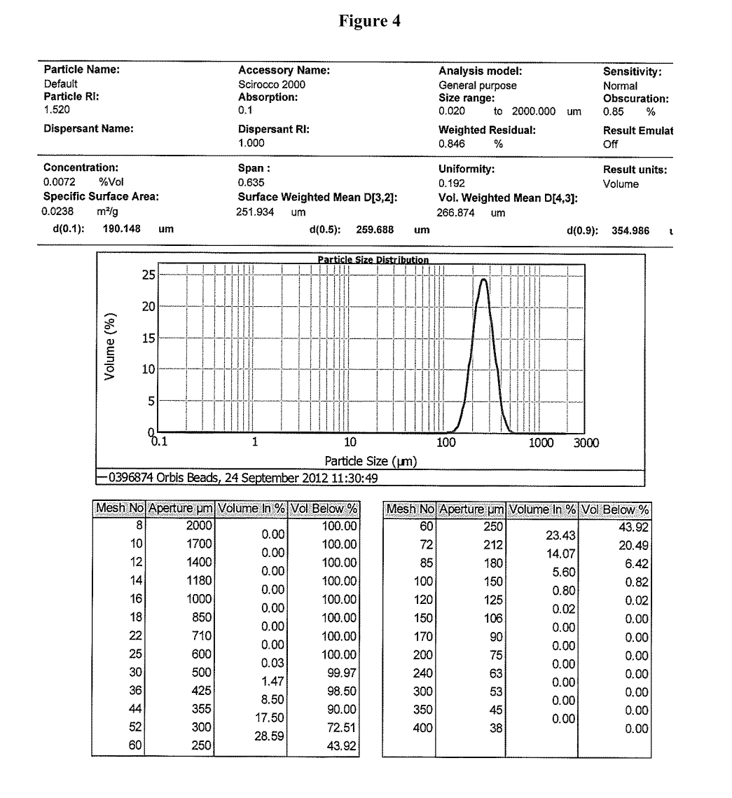

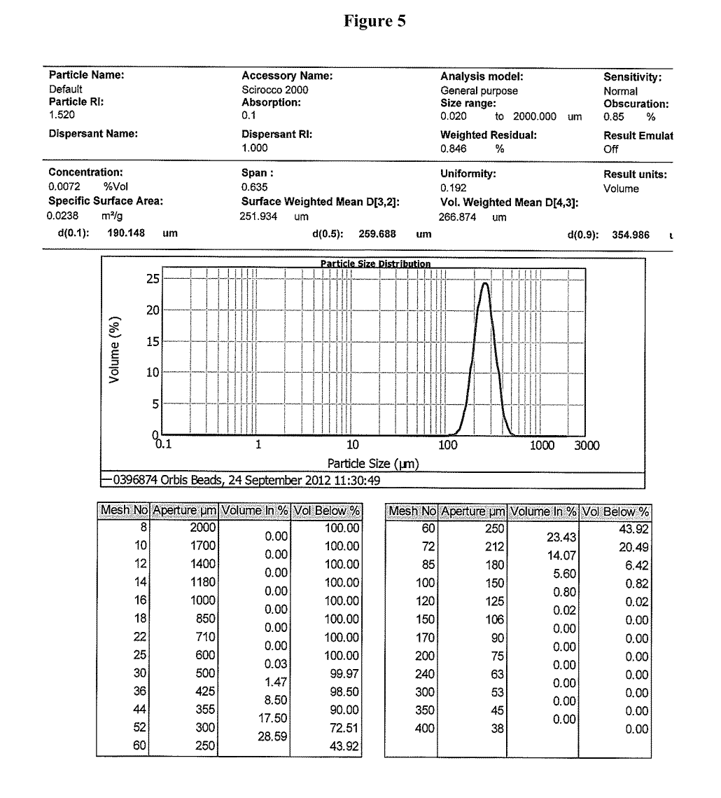

Particles were analyzed for their size distribution using a light-scattering apparatus (Malvern). The results are shown in FIG. 4 and FIG. 5.

### Example 3

### Particles Containing Guaifenesin

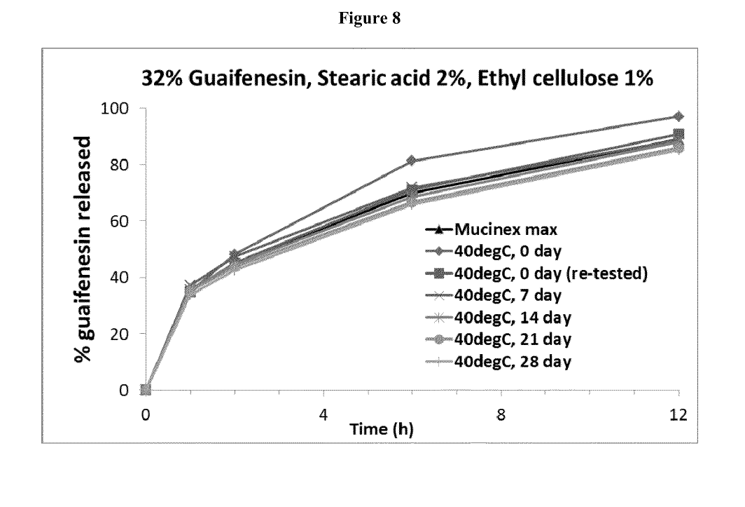

An exemplary guaifenesin particle was formed with 57% carnauba wax, 10% stearic acid, 32% guaifenesin, and 1% ethyl cellulose. The release of guaifenesin from these particles was measured at 40° C. over 28 days, as described above. The results are shown in FIG. 8 and Table 4.

TABLE 4

| | | Percent Release | | | | |
|---|---|---|---|---|---|---|
| | | Day 0 | | | | |
| Time (h) | Day 0 | (Re-tested) | Day 7 | Day 14 | Day 21 | Day 28 |
| 0 | 0.0 | 0.0 | 0.0 | 0.0 | 0.0 | 0.0 |
| 1 | 34.8 | 35.0 | 37.2 | 35.7 | 35.0 | 33.8 |
| 2 | 48.2 | 45.1 | 47.4 | 44.7 | 43.8 | 42.5 |
| 6 | 81.4 | 71.0 | 71.8 | 68.6 | 66.7 | 65.8 |
| 12 | 97.0 | 90.9 | 88.8 | 88.1 | 86.0 | 85.1 |

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A composition comprising particles, the particles comprising guaifenesin, a hydrophobic wax matrix, a stabilizer, and a release modifier; wherein the particles are substantially free of water; and wherein the particles have a diameter of from about 20 μm to about 500 μm.

2. The composition of claim 1, wherein the particles have a diameter of from 100 μm to about 200 μm.

3. The composition of claim 1, wherein the particles are characterized by a standard deviation of 10% or less for a release profile at any given time point during the course of dissolution when placed at 40° C. for up to at least 4 weeks as measured by United States Pharmacopeia (USP) II dissolution.

4. The composition of claim 1, wherein the particles have a diameter with no more than a 25% standard deviation from the mean particle size diameter.

5. The composition of claim 1, wherein the particles have a diameter with no more than a 15% standard deviation from the mean particle size diameter.

6. The composition of claim 1, wherein the particles have a diameter with no more than a 10% standard deviation from the mean particle size diameter.

7. The composition of claim 1, wherein the guaifenesin is present in an amount from about 20% to about 60% by weight of the particles.

8. The composition of claim 1, wherein the hydrophobic wax matrix is chosen from one or more of ceresine wax, beeswax, ozokerite, microcrystalline wax, candelilla wax, montan wax, carnauba wax, paraffin wax, cauassu wax, Japan wax, Shellac wax, and mixtures thereof.

9. The composition of claim 1, wherein the hydrophobic wax matrix is chosen from one or more of candelilla wax and carnauba wax.

10. The composition of claim 1, wherein the hydrophobic wax matrix is present in an amount from about 30% to about 80% by weight of the particles.

11. The composition of claim 1, wherein the stabilizer is chosen from one or more of cellulose, ethyl cellulose, hydroxyproylmethyl cellulose, microcrystalline cellulose, cellulose acetate, cellulose phthalate, methyl cellylose, and mixtures thereof.

12. The composition of claim 1, wherein the release modifier is chosen from one or more of stearic acid, sodium stearate, magnesium stearate, glyceryl monostearate, and cremophor (castor oil).

13. The composition of claim 1, wherein the particles comprise a layer disposed on the surface of the particle.

14. The composition of claim 1, further comprising a liquid vehicle.

15. The composition of claim 1, further comprising a liquid vehicle, wherein the liquid vehicle comprises guaifenesin.

16. The composition of claim 1, further comprising a densifier.

17. A particle consisting essentially of guaifenesin, a hydrophobic wax matrix, a stabilizer, and a release modifier.

18. The composition of claim 17, wherein the guaifenesin is about 32% by weight of the particle, the hydrophobic wax matrix is about 50% to 70% by weight of the particle, the stabilizer is about 1% of the particle, and the release modifier is about 2% by weight of the particle.
